(19) United States
(12) Patent Application Publication
(10) Pub. No.: US 2010/0113948 A1
(43) Pub. Date: May 6, 2010

(54) HEART RATE MEASUREMENT

(75) Inventors: Guang Zhong Yang, Surrey (GB); Benny Ping Lai Lo, London (GB); Lei Wang, London (GB)

Correspondence Address: BROOKS KUSHMAN P.C., 1000 TOWN CENTER, TWENTY-SECOND FLOOR, SOUTHFIELD, MI, 48075, US

(73) Assignee: IMPERIAL INNOVATIONS LIMITED, London (GB)

(21) Appl. No.: 12/531,133

(22) PCT Filed: Mar. 11, 2008
(86) PCT No.: PCT/GB08/00845

§ 371 Date: Sep. 14, 2009

(30) Foreign Application Priority Data
Mar. 15, 2007 (GB) 0705033.9

(51) Int. Cl. A61B 5/02

(52) U.S. Cl. 600/500

(57) ABSTRACT

A reflective photoplethysmograph sensor (for example mounted in an earpiece) arranged for photoplethysmograph measurements behind a subject's ear is provided. Also provided is a wearable photoplethysmograph heart rate sensor which includes a plurality of radiation detectors defining respective sensing planes which are tilted with respect to each other. Further there is provided a photoplethysmograph heart rate sensing system which compensates for motion artefacts using a dark signal which may be derived during an off phase of a duty cycle of an emitter and a photoplethysmograph system arranged to select between a plurality of detectors based on a quality measure. Combinations of the systems and sensors are also disclosed.

The present invention relates to heart rate measurement sensor and system and in particular, although not exclusively, a reflective photoplethysmograph earpiece sensor.

Continuous and non-intrusive monitoring of cardiovascular function has clear applications in pervasive healthcare. Although extensive measurement of biomechanical and biochemical information is available in almost all clinical settings, the diagnostic and monitoring utility is generally limited to brief time points and perhaps unrepresentative physiological states such as supine and sedated, or artificially introduced exercise tests. Transient abnormalities, in this case, cannot always be captured. Many cardiac diseases are associated with episodic rather than continuous abnormalities. These abnormalities are important but their timing cannot be predicted and much time and effort is wasted in trying to capture an “episode” with controlled monitoring. Important and even life-threatening disorders can go undetected because they occur only infrequently and may never be recorded objectively.

Thus far, a range of ECG monitoring devices that permits the continuous recording of heart-rate variability has been proposed. These include digital Holter devices for capturing arrhythmogenic events and chest-strip type devices for professional sports and exercise. Photoplethysmograph (PPG) devices have received significant attention in recent years due to the possibility of integrating them with wearable, pervasive sensing devices. PPG is based on the detection of subcutaneous blood perfusion by shining light through a capillary bed. As arterial pulsations fill the capillary bed, the volumetric changes of the blood vessels modify the absorption, reflection or scattering of the incident light, so the resultant reflected/transmitted light could indicate the timing of cardiovascular events, such as heart rate. A PPG sensor requires at least one light source (usually infrared) and one photo detector in its close proximity. PPG sensors are commonly worn on fingers because of the high signal strength that can be achieved. This configuration, however, is not suitable for pervasive sensing as most daily activities involve the use of fingers.

Different positioning of the PPG sensors has been explored extensively in recent years. This includes body locations such as ring finger, wrist, brachia, belly and oesophagus. For commercial clinical PPG sensors, it is also common to use earlobe and forehead as the anatomical regions of interest. An ear-clip attached to the earlobe can cause pain if it is used over a long period of time, and neither approach is suitable for pervasive sensing applications.

An example of a portable equipment which can be worn on the ear and includes a heart rate measuring device is described in US2003/0233051. The equipment includes an earphone secured to the ear using a horn worn behind the ear. A light source is provided on the horn and an optical sensor on the earphone such that light from the light source is detected by the sensor after passing through the cartilage of the auricula, that is a transmissive PPG arrangement. Problematic for the application to pervasive healthcare is the relatively bulky earphone part of the equipment which needs to be worn outside on the ear. Further, the transmissive design may increase the amount of light needed for PPG and consequently the required driving current. This is further exasperated by the use of two light emitters at separate wavelengths for the purpose of artefact compensation as described in more detail in WO99/32030.

U.S. Pat. No. 5,431,170 is an example of a reflective PPG pulse rate meter which uses a first emitter and receiver at a wavelength such that the corresponding measurements vary with blood or other fluid flow pulsations and a second light emitter and receiver at a different wavelength at which measured signals do not vary with blood or other fluid flow pulsations. The two measurements are compared to cancel out movement or vibration noise for the signal obtained from the light sensor which obtains measurements which vary with blood or other fluid flow pulsations. Again, the use of two separate emitters and receivers increase the number of components, and therefore costs, as well as increasing the power consumption due to the fact that two separate emitters and receivers need to be powered.

A further drawback of the prior art devices described above is that only a single sensor location is provided. In particular in the example of the ear worn device of US2003/0233051 the location of the emitter and receiver is fixed relative to the anatomy of a subject's ear and, accordingly, due to variations of individual anatomy, may not be in an optimal position for some subjects.

The invention is set out in independent claims 1, 7, 14 and 15. Further, optional features are set out in the dependent claims.

In one embodiment, a PPG sensor, which may be wearable behind a subjects ear, is arranged to detect radiation reflected from the cranial surface of the auricula, the adjacent temporal scalp or both. Advantageously, by using radiation reflected from behind the ear, the sensor can be worn entirely behind the ear thus be minimally visible and obstructive. Moreover, the skin portions from which the signals are obtained have rich vascularity (i.e. superficial temporal and posterior auricular arteries/veins and adjunct capillaries) and a thin epidermal layer with relatively little skin pigmentation—this is advantageous because the total optical absorption of the epidermis depends primarily on melanin absorptions such that the PPG radiation reaches the subcutaneous blood vessels with less attenuation for the chosen region of skin.

In a further embodiment, which may be combined with any of the other embodiments, a wearable PPG heart rate sensor includes first and second radiation detectors which are oriented differently with respect to each other and may have corresponding sensing surfaces which define sensing planes tilted with respect to each other, for example by 45° to 135° or, more particularly approximately 90°. One of the planes may be arranged such that the corresponding detector senses radiation from the cranial surface of the auricula and the other one from the adjacent temporal scalp. For optical shielding, the detectors may be recessed into a sensor housing.

In a further embodiment which may be combined with one or more of the other embodiments, a PPG heart rate sensing system includes a PPG sensor which has an emitter and a detector operating at a wavelength suitable for PPG and a data processor configured to derive a heart rate signal from a first signal from the detector when the emitter is on and a second signal from the detector when the emitter is off.

Conveniently, the emitter may be operated in accordance with a duty cycle, for example of 25 percent, and the second signal can be obtained during those parts of the duty cycle when the emitter is off. Advantageously, detecting the second signal during off-periods only marginally increases the power consumption of the system by the amount required for driving the detector. The data processor may be arranged to compare the frequency spectrum of the two signals to determine the peak in the first signal which corresponds to the heart rate. Alternatively, the compensator may derive a filter for the first signal from the frequency spectra of the signals. A heart rate signal may then be determined from a spectral analysis of the first signal after the filter has been applied.

In yet a further embodiment which may be combined with one or more of the other embodiments, a PPG heart rate sensor system includes a PPG sensor having a plurality of detectors each for detecting a PPG signal and a selector arranged to calculate a quality measure for each PPG signal from the respective detectors and to select one of the detectors based on the quality measure, the system being arranged to derive a heart rate signal from the selected detector. Advantageously, this allows the detector giving the best signal to be selected for the measurement thereby accounting for variations in the anatomy between subjects.

For example, the quality measure may be a measure comparing the energy in a frequency band around a detected heart rate frequency to the total energy in the signal. For example, the selection may be made initially during a calibration phase, periodically at pre-determined intervals during measurement or when a drop of the quality measure below a threshold or a sufficiently large change of the measure is detected.

The PPG heart rate measurement system may include a sensor as described above and may be housed in a housing wearable behind a subjects ear which further may house a wireless transmitter for transmitting a heart rate signal to a receiver.

Embodiments are now described by way of example only and with reference to the accompanying drawings in which:

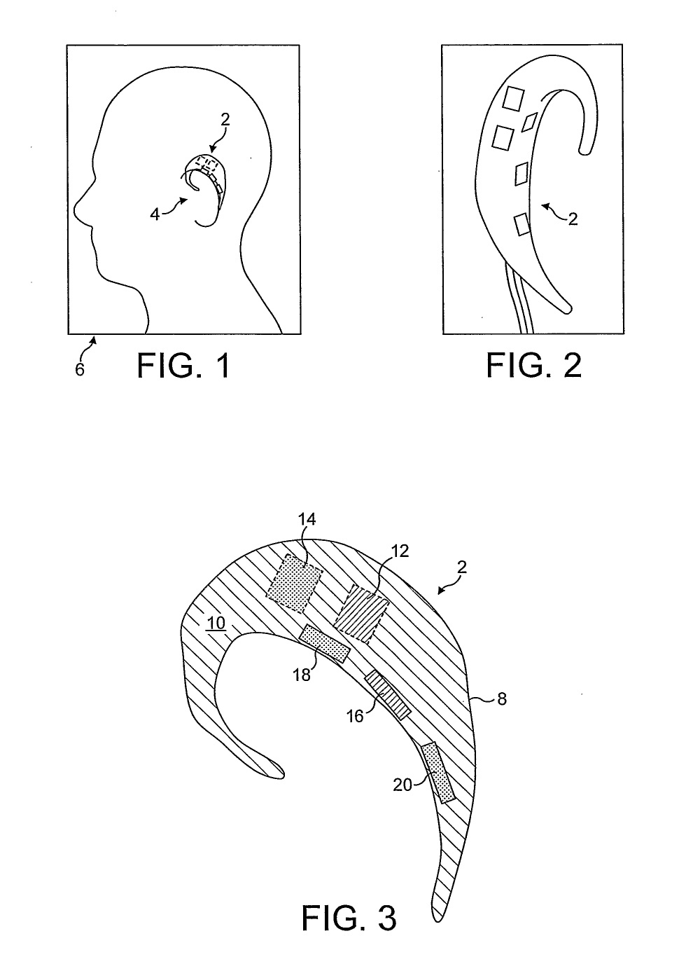

FIG. 1 schematically shows a subject wearing a wearable heart rate sensor behind the ear;

FIG. 2 shows a wearable sensor in accordance with one embodiment;

FIG. 3 shows a schematic cross-sectional view of the wearable heart rate sensor;

FIG. 4 is a block diagram of a heart rate measuring system; and

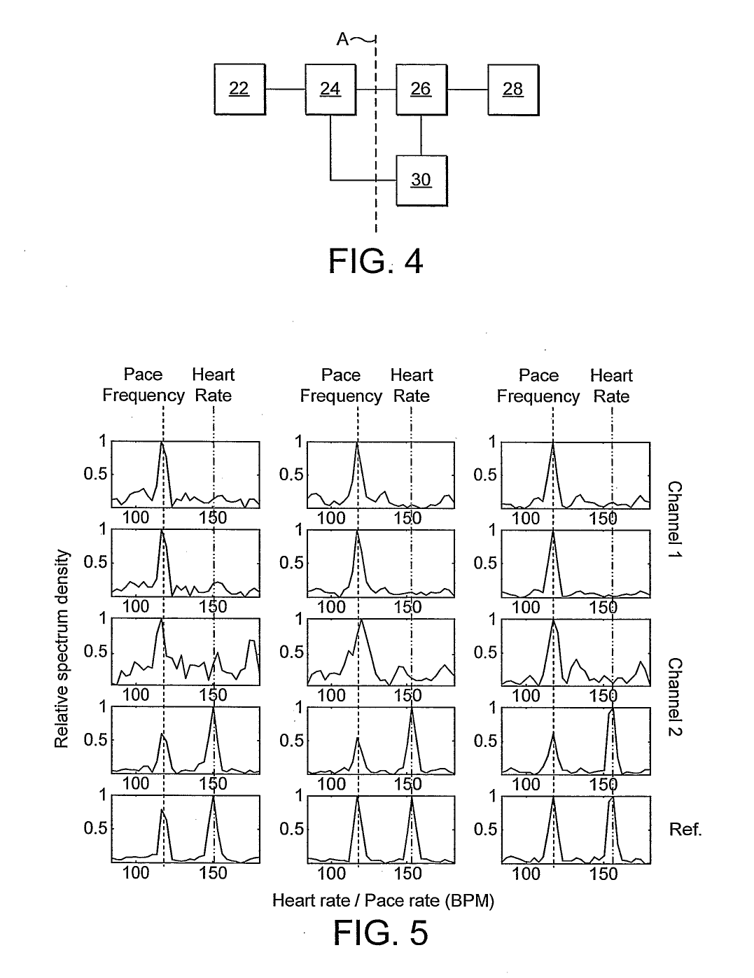

FIG. 5 depicts signals recorded using the heart rate measuring system and a reference signal.

With reference to FIGS. 1, 2 and the cross-sectional view in FIG. 3, a wearable sensor 2 which can be worn behind the ear 4 of a subject 6 includes a housing 8 of a shape such that it can be worn as an ear piece behind the ear. Recessed about 1 millimetre into a temporal surface 10 is a temporal light emitter 12 and a temporal light detector 14 arranged to, respectively, irradiate the subjects temporal scalp and receive reflected radiation therefrom. An auricular light emitter 16 faces the auricula when the wearable sensor is worn by the subject. A first auricular light detector 18 and a second auricular light detector 20 are located either side of the auricular light emitter 16. The auricular emitters and detectors are arranged to, respectively, irradiate and receive radiation from the cranial surface of the auricula when the sensor is worn by the subject. The first auricular detector 18 detects radiation reflected from a superior cranial auricular region and the second auricular detector 20 detects radiation from a region inferior and anterior to the first auricular detector 18.

The temporal detector 14 and each of the auricular detectors 18 and 20 each define a sensing plane by their sensitive surface and from the above description it will be clear that the sensing surface of the temporal detector 14 is tilted with respect to the sensing planes defined by the auricular detectors 18 and 20, depending on the exact geometry of the housing, by between 45° and 135°, for example approximately 90°. Furthermore, the sensing planes of the auricular detectors 18 and 20 are also tilted with respect to each other. Advantageously, because the three detectors are located in different locations and at different orientations, signals from anatomically distinct regions may be recorded, thereby increasing the likelihood of obtaining a good signal from one of the detectors. For example, the three signals may be averaged together or, alternatively the detector which provides the best signal for a given subject (which will vary due to anatomical variations between subjects) can be selected for data collection, as described in more detail below.

The light emitters 12 and 16 may be light emitting diodes, for example DLED-690/905, DLED-690/940 from UDT® and PDI-E835 from API®. The former two provide both visible red and infrared radiation but, in one embodiment, only the infrared radiation channel is used. Detectors 14, 18 and 20 may include photo diodes such as PIN-4.0 or PIN-8.0 from UDT® or BPW34F from Siemens®. The active areas of these photo diodes were 4, 8 and 7 mm², respectively. While the latter photo diode includes a daylight filter, use of the daylight filter was not found to significantly influence performance. The distances between the emitters and corresponding detectors may be in a range of 8 to 12 mm. The recessing of the emitting and detecting components provides some degree of optical shielding to avoid cross-talk. The non-sensitive side of the sensor is painted black to prevent multiple scatterings.

With reference to FIG. 4, the emitters and detectors are schematically represented by block 22 and are driven by respective interface circuitry indicated at block 24. The interface circuitry 24 generally drives the emitters and conditions signals from the sensors. In one embodiment, it includes a current regulating diode in series with each emitter, for example a SST50X current regulating diode from Vishay®. The emitter driving current is set by the current regulating diode and, in one embodiment, driving currents between 4 to 8 mA are appropriate. Output currents from the detectors are fed, in one embodiment, into differential trans-impedance amplifiers, for example OP297s from Analog®, together with a +/−3V power supply from National Semiconductors®. In an alternative embodiment, a rail-to-rail amplifier LT1491 from Linear® may be used for a different gain level. The interface circuitry 24 is provided with three amplification channels, one for each detector to allow for a simultaneous data collection. Average power consumption is approximately 6 mW per channel. In yet a further embodiment, an integrated driving circuit as disclosed in Wong A, Pun K P, Zhang Y Z et at (2005) A near-Infrared heart rate measurement IC with very low cutoff frequency using current steering technique. IEEE Trans. On Circuits and Systems-I Regular Papers 52(12): 2642-2647, incorporated herewith by reference herein, may be used.

In one embodiment the sensor (detector and emitter) 22 and interface 24 circuitry are provided within the housing 8 with the remaining components provided remotely and connected by a wired link as indicated by dashed line A. In that embodiment, the output from the amplifiers within interface circuitry 24 is provided to a PC or other computing platform via a digital acquisition device, for example USB-6009 from National Instruments® at an initial sampling rate of, for example, 1 kHz per channel. Data processing (and visualisation if required) may then be completed online or offline, as appropriate, down sampling the signal as required.

In another embodiment, the data processor 26, as well as a wireless link 28 (although a wired link may equally be used) and channel selector 30 (to be described in detail below) are housed within the housing 8. In this embodiment the data processor may include a Texas Instruments® MSP430 16-bit ultra low power RISC processor with 60 KB+256 B Flash memory, 2 KB RAM, 12-bit ADC, and 6 analog channels (connecting up to 6 sensors). In this embodiment, as there are three detectors and corresponding amplifiers, a further three channels are available for other data sources, for example a three axis accelerometer. Such an accelerometer can be used to provide data which could be used in correcting artefacts in the PPG signals due to movement, as described in European patent application no. 01203686.9 filed 28 Sep. 2001, herewith incorporated by reference herein. The acceleration sensor may further be used for activity recognition, for example gate analysis as described in co-pending patent application no. PCT/GB2007/000358 entitled Gait Analysis and having the same Applicant/Assignee as the present application, herewith incorporated by reference herein.

In one embodiment, the acceleration sensor (or another motion sensor) may be used to infer the level of activity of a subject wearing the sensor. An analysis of the acceleration sensor outputs is used in this embodiment to time stamp automatically different states of physical exercise such as rigorous exercise (acceleration signals on average above a threshold, for example) or rest (acceleration signals on average below a threshold, for example). This could be used, for example, for recovery measurement. A change from exercising at a high level to rest is time stamped in this example and the time taken for the heart rate to return to a normal resting rate is measured.

Additionally, the housing 8 houses a wireless module 28 with a throughput of 250 KBPS and a range over 50 m. A 512 KB serial flash memory may further be incorporated for data storage or buffering. The data processor 26 may run TinyOS by U.C. Berkeley which is a small, open source and energy efficient sensor port operating system.

The data processor 26 is configured to determine a subject's heart rate from the PPG signal measured by the detector by identifying a peak in the frequency spectrum of the detector signal as corresponding to the heart rate, as described in more detail in Webster J G (1997) Design of pulse oximeters. Institute of Physics Publishing. Specifically, in one embodiment, the PPG signal captured by the detector is down-sampled to 50 samples per channel (if necessary) followed by baseline (D.C.) subtraction and band-pass filtering with a pass band of 0.5 Hz to 4 Hz, either using a digital filter or an additional analog component. Frequency spectra may be calculated using a moving-window Fast Fourrier Transform (Hanning-windowed, window length 20 seconds), for example.

In one embodiment, the data processor 26 is configured to implement an artefact, for example due to motion, compensation algorithm. In general, the emitter/detector 22 and driving 24 circuits do not operate continuously but rather intermittently, for example with a duty cycle of 25 percent (other duty cycles, for example in the range of 10% to 50% are equally envisaged). For example, the circuits may become active for 250 ms in every second. The disclosed compensation algorithm uses a signal measured while the emitter is off (and, of course, the corresponding detector is active) to measure a signal used in compensating the PPG signal measured by the detector while the emitter is inactive by detecting reflected ambient light without the need for a further emitter as in the prior art. This reduces the number of components and also the overall current consumption as only the amplifying current is required to obtain the signal. Effectively, the algorithm makes use of a “dark signal” to correct for artefacts, for example motion artefacts.

In one particular implementation, the frequency spectrum obtained for the PPG signal is compared to the frequency spectrum of the dark signal to determine the spectral peak corresponding to heart rate. This can be understood with reference to FIG. 5 in which the first row of each channel shows the spectrum corresponding to the dark signal and a second row of each channel shows the spectrum for the PPG signal, the last row showing the spectrum for a signal recorded using a commercial bedside pulse oximeter (OxiMax N-560 from Nellcor,®). As can be seen from the graphs for channel 2 in FIG. 5, the dark signal has a spectral peak at 115 hertz, the step frequency at which the signals were recorded while the PPG signal has a second peak at the heart rate frequency of 150, 155 and 160 beats per minute from left to right.

Accordingly, in one approach, peaks are detected in both the dark signal and the PPG signal and only that peak which is present in the PPG signal but not in the dark signal is attributed to the heart rate and a heart rate measurement at the peak frequency is established.

In an alternative implementation, a step or artefact frequency is derived from the dark signal and the step frequency band is then removed from the PPG signal using a notch filter to remove a frequency band centred on the step frequency and, for example, of width 0.2 Hz or +/−6 beats per minute. This substantially suppresses the step frequency peak and leaves the heart rate frequency peak to be measured to obtain the heart rate.

Once the heart rate signal is calculated it may either be stored on a suitable storage medium, displayed on a display screen, or, where appropriate, transmitted to a receiver using the wireless link 28.

In yet a further embodiment, if the system includes an acceleration sensor (or other motion sensor), as described above, the acceleration sensor may be used to cross-check the motion-related peak in the spectrum of the dark and PPG signals. If the step frequency is close to the heart rate, the corresponding peak in the PPG and dark signal spectrum will be overlapping with the heart rate peak in the PPG signal. By calculating a principal motion frequency from the acceleration sensors as an independent signal source, it can be verified that the observed spectrum of the PPG and dark signals is due to the heart rate and step frequency being close (as explained above) rather than due to a system failure. Comparison with the principle motion frequency therefore allows to determine the reliability of the heart rate signal derived from the PPG and dark signals. Of course, the acceleration signal may also be used directly to identify the heart rate peak in the PPG spectrum.

In the exemplary subject data in FIG. 5, it is clear that, while channel 2 has a clear peak corresponding to heart rate in the PPG signal, no such peak is detected in the PPGs signal from channel 1 (channel 1 corresponding to detector 14 and channel 2 corresponding to detector 18). It is generally observed that one of the three channels tends to provide a better signal in a given subject but that this channel varies between subjects, presumably due to anatomical variations between subjects.

For the best available signal to be used for heart rate measurement, a channel selection algorithm and a corresponding channel selector 30 is implemented by data processor 26. A quality measure is calculated for each of the three channels/detectors during a calibration phase and a signal of a detector selected based on the quality measure, for example the channel with the best quality measure, is then used to calculate a heart rate.

The calibration phase may be implemented once as an initialisation when the sensor is started or it may be entered periodically at predetermined intervals, for example every five minutes. Yet a further possibility is to enter the calibration phase when a quality measure of the selected channel drops below a predetermined threshold or if a change in the quality measure larger than a certain value is detected.

In one embodiment, the channel selector 30 is operatively coupled to the driving circuit 24 such that, outside the calibration phase, only the detector and amplifier of the selected channel and the corresponding emitter are active, thereby achieving further power savings.

In one implementation, a suitable quality measure may be a heart rate spectrum fidelity index F_{HRS}, defined as the ratio of the energy within a frequency band centred on the heart rate to the total energy of the spectrum. This index has a positive value between 0 and 1, for example for a single-frequency sine wave at the heart rate frequency F_{HRS}=1 and for white noise F_{HRS} is equal to the ratio of the frequency band over half the sampling rate (see Celka P, Verjus C, Vetter R et al (2004) Motion resistant earphone located infrared based heart rate measurement deice, Proc. 2^{nd} International Conference Biomedical Engineering, Innsbruck, Austria, 2004, pp 582-585, incorporated herewith by reference herein). In one particular example, the frequency band used for the calculation of F_{HRS} was set to be 0.2 Hz.

It will be understood that the above description is by way of example only and that various modifications, alteration and juxtapositions of the subject matter disclosed will be apparent to the skilled person and are intended to be covered within the scope of the appendent claims.

1. A photoplethysmograph heart rate sensing system which includes a photoplethysmograph sensor having an emitter for emitting radiation at a wavelength suitable for photoplethysmography and a detector for detecting radiation emanating from the light source and reflected by a region of skin from which a photoplethysmographic signal is being measured and a data processor configured to derive a heart rate signal from a first signal from the detector when the emitter is on and a second signal from the detector when the emitter is off.

2. A system as claimed in claim 1 in which the data processor is configured to determine a spectrum for each of the respective signals and to determine a peak in the spectrum of the first signal associated with the heart rate by comparing the spectra.

3. A system as claimed in claim 1 in which the data processor is configured to determine a spectrum for each of the respective signals and to derive a notch filter to suppress a motion-related peak in the first signal from the spectra and the system is configured filter the first signal using the derived filter and to use the filtered signal to determine a heart rate.

4. A system as claimed in claim 1, in which the emitter operates on a duty cycle, the second signal being obtained while the emitter is off during the duty cycle.

5. A system as claimed in claim 1 in which the duty cycle is between 10% to 50%, for example 25%.

6. A system as claimed in claim 1, which includes a photoplethysmograph sensor having a plurality of detectors each for detecting a photoplethysmograph signal and a selector arranged to calculate a quality measure for each signal from the respective detectors and to select one of the detectors based on the quality measure, the system being arranged to determine a heart rate signal from the selected detector.

7. A photoplethysmograph heart rate sensing system which includes a photoplethysmograph sensor having a plurality of detectors each for detecting a photoplethysmograph signal and a selector arranged to calculate a quality measure for each signal from the respective detectors and to select one of the detectors based on the quality measure, the system being arranged to determine a heart rate signal from the selected detector.

8. A system as claimed in claim 7 in which the quality measure includes a ratio of the spectral energy within a frequency band centred on a heart rate frequency to the total spectral energy.

9. A system as claimed in claim 7 in which the selector is arranged to calculate the quality measure during a calibration phase.

10. A system as claimed in claim 9 in which the selector is arranged to enter the calibration phase once during initialisation of the system, periodically at predetermined intervals, when the quality measure drops below a predetermined threshold or when the quality measure changes by more than a predetermined value.

11. A system as claimed in claim 7 in which the selector is arranged to power only the selected detector and an associated emitter once a detector is selected.

12. A system as claimed in claim 1 which includes a motion sensor, for example an accelerometer, and means for detecting an activity state based on a signal of the motion sensor to timestamp the heart rate signal or trigger a heart rate measurement.

13. A system as claimed in claim 1 which includes means for determining a principle motion frequency from a motion sensor, for example an accelerometer, and using the determined principle motion frequency to determine the reliability of the heart rate signal derived from the first and second signals.

14. A photoplethysmograph heart rate sensor which includes a plurality of radiation detectors oriented differently with respect to each other.

15. A photoplethysmograph heart rate sensor which is arranged to detect radiation reflected from the cranial surface of a subject's auricula, the adjacent temporal scalp or both.

16. A sensor as claimed in claim 14 which is wearable behind the subject's ear.

17. A sensor as claimed in claim 14, which includes a first radiation detector having a sensing surface defining a first plane and a second radiation detector having a sensing surface defining a second plane which is tilted with respect to the first plane.

18. A sensor as claimed in claim 17 in which the planes are tilted with respect to each other by an angle of 45 degrees to 135 degrees, in particular approximately 90 degrees.

19. A sensor as claimed in claim 17 in which the first radiation detector is arranged to detect radiation reflected from the cranial surface of the auricula and the second radiation detector is arranged to detect radiation reflected from the adjacent temporal scalp.

20. A sensor as claimed in claim 17 in which the first plane is generally extending away from the temporal scalp and the second plane is generally parallel with the temporal scalp when the sensor is worn behind a subject's ear.

21. A sensor as claimed in claim 14 in which the detectors are recessed within a sensor housing, thereby providing optical shielding between the detectors.

22. A system as claimed in claim 1 including a photoplethysmograph sensor which includes a plurality of radiation detectors oriented differently with respect to each other.

23. A system as claimed in claim 1 which is housed in a housing wearable behind a subject's ear.

24. A system as claimed in claim 23 which further includes a wireless transmitter for transmitting the heart rate signal to a receiver.
